(19) United States
(12) Patent Application Publication
(10) Pub. No.: US 2026/0049326 A1
(43) Pub. Date: Feb. 19, 2026

(54) APPLICATION METHOD IN RICE CALLUS DIFFERENTIATION BASED ON ORYZA SATIVA LEAFY COTYLEDON 1 GENE

(71) Applicant: Hainan Institute of Zhejiang University, Sanya (CN)

(72) Inventors: Fu Guo, Sanya (CN); Wenzhuo Zeng, Sanya (CN); Qingyao Shu, Sanya (CN)

(21) Appl. No.: 19/298,286

(22) Filed: Aug. 13, 2025

(30) Foreign Application Priority Data
Aug. 13, 2024 (CN) 202411103031.X

(51) Int. Cl. C12N 15/82; C12N 9/22; C12N 15/11

(52) U.S. Cl. CPC C12N 15/8262; C12N 9/226; C12N 15/11; C12N 15/8205; C12N 15/8213; C12N 2310/20

(57) ABSTRACT

The present disclosure provides an application method in rice callus differentiation based on an Oryza sativa Leafy Cotyledon 1 (OsLEC1) gene, including the steps of: selecting guide ribonucleic acid (gRNA) target sites; cloning tandem fragments including gRNA; ligating each gRNA fragment; performing polymerase chain reaction (PCR) amplification on a ligation product; performing enzyme digestion on the purified product and a target vector; transforming the ligated vector; performing Agrobacterium-mediated genetic transformation of rice; and screening and identifying transgenetic plants. In the present disclosure, the differentiation of callus directly affects the emergence efficiency of transgenic plants. The knockout of OsLEC1 can promote the differentiation of rice callus, suggesting that OsLEC1 can serve as an important target gene for improving the transformation efficiency of rice and even gramineous crops. OsLEC1 can be used as a starting point to construct various molecular tools to enhance transformation efficiency.

### CROSS REFERENCE TO RELATED APPLICATION

This patent application claims the benefit and priority of Chinese Patent Application No. 202411103031.X filed with the China National Intellectual Property Administration on Aug. 13, 2024, the disclosure of which is incorporated by reference herein in its entirety as part of the present application.

### REFERENCE TO SEQUENCE LISTING

A computer readable XML file entitled “HKIP-US-1-1312-22-Sequence Listing”, that was created on Aug. 11, 2025, with a file size of about 23,101 bytes, contains the sequence listing for this application, has been filed with this application, and is hereby incorporated by reference in its entirety.

### TECHNICAL FIELD

The present disclosure relates to the technical field of rice callus differentiation, and in particular to an application method in rice callus differentiation based on an Oryza sativa Leafy Cotyledon 1 (OsLEC1) gene.

### BACKGROUND

Plant tissue culture technology, which emerged in the early 20th century, refers to the process of culturing isolated plant organs, tissues, cells, protoplasts, etc. in vitro under suitable conditions under aseptic conditions, inducing callus, adventitious buds, etc., and further forming a complete plant.

In order to improve the differentiation rate of rice callus, researchers have adopted a series of innovations, including drying treatment, radiation treatment, different hormone ratios, adding different carbon sources, adding elements including copper and silver, adding proline, adding paclobutrazol (MET) and adding activated carbon. Drying treatment and radiation treatment are time-consuming, the radiation treatment method also requires specific instruments, and adding elements including copper and silver causes additional heavy metal pollution. In short, the above methods are numerous and messy, and some methods only have significant effects on certain plant varieties, which are not suitable for rice and are difficult to meet the needs of workers.

### SUMMARY

In order to solve the above technical problems, an application method in rice callus differentiation based on an OsLEC1 gene is provided, and the technical solutions solve the problem that in order to improve the differentiation rate of rice callus, researchers have adopted a series of innovations, including drying treatment, radiation treatment, different hormone ratios, adding different carbon sources, adding elements including copper and silver, adding proline, adding MET and adding activated carbon. Drying treatment and radiation treatment are time-consuming, the radiation treatment method also requires specific instruments, and adding elements including copper and silver causes additional heavy metal pollution. In short, the above methods are numerous and messy, and some methods only have significant effects on some specific plant varieties, but are not suitable for rice problems.

In order to achieve the above objective, the technical solutions adopted by the present disclosure are as follows.

The application method in rice callus differentiation based on an OsLEC1 gene includes the steps of:
S1, selecting guide ribonucleic acid (gRNA) target sites, the sequence of the target sites being located on the OsLEC1 gene, and the OsLEC1 gene being Sequence I recorded in this invention
S2, cloning tandem fragments including gRNA;
S3, ligating each gRNA fragment;
S4, performing polymerase chain reaction (PCR) amplification on a ligation product;
S5, performing enzyme digestion on the purified product and a target vector;
S6, transforming the ligated vector;
S7, performing Agrobacterium-mediated genetic transformation of rice; and
S8, screening and identifying transgenetic plants.

Preferably, the selecting gRNA target sites specifically includes the steps of:
following target site design principles for clustered regularly interspaced short palindromic repeats-CRISPR-associated protein 9 (CRISPR-Cas9) technology; and
designing two gRNA target sites on a gene sequence close to a 5′ end of the OsLEC1 gene.

Preferably, the cloning tandem fragments including gRNA specifically includes the steps of:
using plasmid pGTR as a template, and amplifying three fragments L1, L2, and L3 including gRNA sequences by PCR; and
taking 5-10 μL of products, detecting the products by 1% agarose gel electrophoresis, purifying and recovering a target fragment, and determining the three PCR products L1, L2 and L3.

Preferably, the ligating each gRNA fragment specifically includes the steps of:
mixing the 3 fragments in equal amounts according to concentrations of the determined PCR products; and
adding T7 ligase and BsaI enzyme, and performing a reaction in a PCR instrument.

Preferably, the T7 ligase and BsaI enzyme are reacted in the PCR instrument at 37° C. for 5 min and at 20° C. for 10 min, and cycled for 30-50 times.

Preferably, the performing PCR amplification on a ligation product specifically includes the steps of:
taking, after the ligation reaction is completed, 1 μL of the ligation product, and diluting the ligation product with 19 μL of water;
using the diluted product was as a template, and performing PCR amplification with primers at two ends; and
taking, after the PCR is completed, 5 μL of the product for electrophoresis detection, with a product size of 500 bp, and purifying the product.

Preferably, the performing enzyme digestion on the purified product and a target vector specifically includes the steps of:
digesting the purified PCR product with FokI, and digesting the target vector pRGEB32 with BsaI; and
mixing, after the digested products are recovered separately, the purified PCR product and the target vector recovered after digestion in equal amounts and ligating the same with T4 ligase at 4° C. overnight.

Preferably, the transforming the ligated vector specifically includes the steps of:
transforming the ligated vector into competent cells of Escherichia coli, smearing, and staying overnight at 37° C.;
picking a single colony for bacterial culture, and identifying whether the target fragment is ligated into the vector by PCR; and
identifying the correctness through sequencing after plasmid extraction, and electroporating the correctly sequenced plasmid into Agrobacterium EHA105.

Preferably, the performing Agrobacterium-mediated genetic transformation of rice specifically includes the steps of:
using the mature embryo callus of wild-type (WT) rice Nipponbare as explants;
performing infection transformation with Agrobacterium EHA105; and
obtaining the transgenetic plants through resistance screening, tissue differentiation, rooting and seedling refining.

Preferably, the screening and identifying transgenetic plants specifically includes the steps of:
extracting the genomic DNA of transgenetic plants, and designing primers on two sides of two gRNA sequences;
amplifying the target fragment by PCR, and detect the mutant by agarose gel electrophoresis and polyacrylamide gel electrophoresis (PAGE); and
purifying and recovering PCR products from mutant strains, and ligating into T-vectors for sequencing, and obtaining mutant materials with knocked-out Oryza sativa AUXIN SIGNALING F-BOX PROTEIN 4 (OsAFB4) gene in rice.

Compared with the related art, the present disclosure provides the application method in rice callus differentiation based on an OsLEC1 gene, and has the following beneficial effects.

In the present disclosure, the differentiation of callus directly affects the emergence efficiency of transgenic plants. The knockout of OsLEC1 can promote the differentiation of rice callus, suggesting that OsLEC1 may serve as an important target gene for improving the transformation efficiency of rice and even gramineous crops. OsLEC1 can be used as a starting point to construct various molecular tools to enhance transformation efficiency.

### BRIEF DESCRIPTION OF THE DRAWINGS

FIG. 1A shows differences in gene sequences of OsLEC1 between Oslec1 mutants and a WT;

FIG. 1B shows differences in protein sequences of OsLEC1 between the Oslec1 mutants and the WT;

FIG. 2A shows photographs of regenerated buds from callus tissues of the Oslec1 mutants and the WT after 30 days of differentiation;

FIG. 2B shows the statistical count of the regenerated buds from the callus tissues of the Oslec1 mutants and the WT after 30 days of differentiation.

### DETAILED DESCRIPTION

The following description is intended to disclose the present disclosure and enable those skilled in the art to realize the present disclosure. The preferred embodiments in the following description are only examples, and other obvious variations can occur to those skilled in the art.

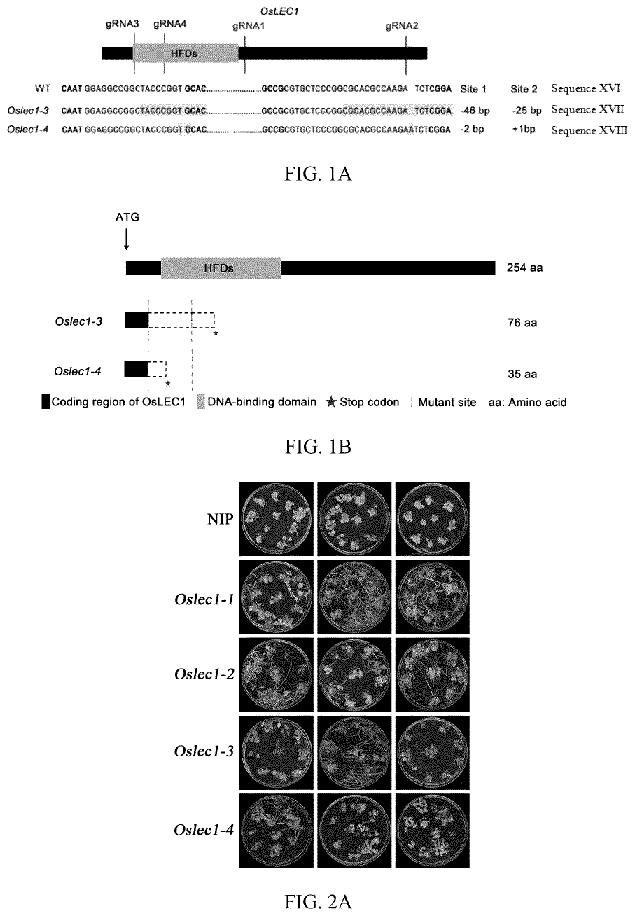

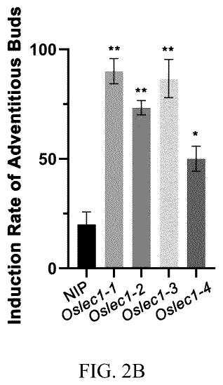

Referring to FIGS. 1A-2B, an application method in rice callus differentiation based on an OsLEC1 gene includes the following steps.

In S1, gRNA target sites were selected, the sequence of the target sites was located on the OsLEC1 gene, and the OsLEC1 gene was Sequence I recorded in this invention

In S2, tandem fragments including gRNA were cloned.

In S3, each gRNA fragment was ligated.

In S4, PCR amplification was performed on a ligation product.

In S5, enzyme digestion was performed on the purified product and a target vector.

In S6, the ligated vector was transformed.

In S7, Agrobacterium-mediated genetic transformation of rice was performed.

In S8, transgenetic plants were screened and identified.

Tandem fragments including gRNA were cloned, specifically including the following steps.

Target site design principles for CRISPR-Cas9 technology were followed.

Two gRNA target sites were designed on a gene sequence close to a 5′ end of the OsLEC1 gene.

It can be understood by those skilled in the art that the OsLEC1 gene is located on chromosome 2 of the rice genome. According to the target site design principles of CRISPR-Cas9 technology, in the present disclosure, the gRNA target sites are designed within exon regions and specifically positioned at the 5′ end of the OsLEC1 gene. The designed primer sequences are as follows.

L1-F:
(Sequence IV)
CGGGTCTCAGGCAGGATGGGCAGTCTGGGCAACAAAGCACCAGTGG
LI-R:
(Sequence V)
CGGGTCTCAGCGTGCGCCGGGTGCACCAGCCGGG
L2-F:
(Sequence VI)
TAGGTCTCCACGCCAAGATCTGTTTTAGAGCTAGAA
L2-R:
(Sequence VII)
CGGGTCTCATAGCCGGCCTCCTGCACCAGCCGGG
L3-F:
(Sequence VIII)
TAGGTCTCCGCTACCCGGGCAGTTTTAGAGCTAGAA
L3-R:
(Sequence IX)
TAGGTCTCCAAACGGATGAGCGACAGCAAACAAAAAAAAAAGCACCGACT
CG

Tandem fragments including gRNA were cloned, specifically including the following steps.

Plasmid pGTR was used as a template, and three fragments L1, L2, and L3 including gRNA sequences were amplified by PCR.

5-10 μL of products were taken, the products were detected by 1% agarose gel electrophoresis, a target fragment was purified and recovered, and the three PCR products L1, L2 and L3 were determined.

It can be understood by those skilled in the art that the sizes of the L1, L2 and L3 products are about 130 bp, 200 bp and 150 bp, and that the PCR reaction system and PCR procedure for amplifying the L1-3 fragment are as follows.

PCR reaction system

| Reagents | Volume (μL) |
|---|---|
| KOD enzyme | 1 |
| 2 × Buffer | 25 |
| L-F | 2.5 |
| L-R | 2.5 |
| Three-distilled water | 8 |
| dNTPs | 10 |
| Template (pGTR) | 1 |
| Total | 50 |

PCR procedure

| Temperature | Time | Cycle number |
|---|---|---|
| 95° C. | 2 min | 1 |
| 98° C. | 10 s | 33 |
| 60° C. | 30 s | |
| 68° C. | 30 s | |
| 68° C. | 7 min | 1 |

Each gRNA fragment was ligated, specifically including the following steps.

The 3 fragments were mixed in equal amounts according to concentrations of the determined PCR products.

T7 ligase and BsaI enzyme were added, and a reaction was performed in a PCR instrument.

The T7 ligase and BsaI enzyme were reacted in the PCR instrument at 37° C. for 5 min and at 20° C. for 10 min, and cycled for 30-50 times.

PCR reaction system

| Reagents | Volume (μL) |
|---|---|
| 2 × T7 ligase buffer | 10 |
| BsaI-HF | 1 |
| T7 ligase | 0.5 |
| L1 | 2 |
| L2 | 2 |
| L3 | 2 |
| Three-distilled water | 2.5 |
| Total volume | 20 |

PCR procedure

| Temperature | Time | Cycle number |
|---|---|---|
| 37° C. | 5 min | 50 |
| 20° C. | 10 min | |

PCR amplification was performed on a ligation product, specifically including the following steps.

After the ligation reaction is completed, 1 μL of the ligation product was taken and diluted with 19 μL of water.

The diluted product was used as a template, and PCR amplification was performed with primers at two ends.

After the PCR is completed, 5 μL of the product was taken for electrophoresis detection, with a product size of 500 bp, and the product was purified.

S1-F:
(Sequence X)
CG GGTCTC A GGCA GGATG GGCAGTCTG GGCA
S1-R:
(Sequence XI)
TA GGTCTC C AAAC GGATG AGCGACAGC AAAC

PCR reaction system

| Reagents | Volume (μL) |
|---|---|
| Diluted GG product | 2.5 |
| S1-F | 2.5 |
| S1-R | 2.5 |
| 2 × TaqMix | 25 |
| Three-distilled water | 17.5 |
| Total volume | 50 |

PCR procedure

| Temperature | Time | Cycle number |
|---|---|---|
| 95° C. | 2 min | 1 |
| 98° C. | 10 s | 33 |
| 60° C. | 30 s | |
| 68° C. | 30 s | |
| 68° C. | 7 min | 1 |

Enzyme digestion was performed on the purified product and a target vector, specifically including the following steps.

The purified PCR product was digested with FokI, and the target vector pRGEB32 was digested with BsaI.

After the digested products are recovered separately, the purified PCR product and the target vector recovered after digestion were mixed in equal amounts and ligated with T4 ligase at 4° C. overnight.

| Reagents | Volume (μL) |
|---|---|
| GG ligation products | 5 μg |
| FokI | 5 |
| Buffer (CutSmart) | 5 |
| Three-distilled water | Make up to 50 |
| Total volume | 50 |
| pRGEB32 vector | 5 μg |
| BsaI | 5 |
| Buffer (CutSmart) | 5 |
| Three-distilled water | Make up to 50 |
| Total volume | 50 |

Ligation of GG Ligation Product after Enzyme Digestion to Target Vector pRGEB32

| Reagents | Volume (μL) |
|---|---|
| GG ligation product after enzyme digestion | 50 ng |
| Vector pRGEB32 after enzyme digestion | 50 ng |
| T4 DNA ligase | 1 |
| 10 × T4 ligase buffer | 1 |
| Three-distilled water | Make up to 10 |
| Total volume | 10 |

The ligated vector was transformed, specifically includes the following steps.

The ligated vector was transformed into competent cells of Escherichia coli, smeared, and stayed overnight at 37° C.

A single colony was picked for bacterial culture, and PCR was used to identify whether the target fragment is ligated into the vector.

The correct extracted plasmid was identified and sequenced, and the plasmid with correct sequencing result was transformed into Agrobacterium EHA105.

PCR identification and sequencing primer sequences are as follows:

U3-F:
(Sequence XII)
AGTACCACCTCGGCTATCCACA
UGW-gRNA-R:
(Sequence XIII)
CGCGCTAAAAACGGACTAGC

Agrobacterium-mediated genetic transformation of rice was performed, specifically including the following steps.

The mature embryo callus of WT rice Nipponbare was used as explants.

Infection transformation was performed with Agrobacterium EHA105.

The transgenetic plants were obtained through resistance screening, tissue differentiation, rooting and seedling refining.

Transgenetic plants were screened and identified, specifically including the following steps.

Genomic DNA of transgenetic plants was extracted, and primers were designed on two sides of two gRNA sequences.

PCR amplification was performed on the target fragment, and mutants were detected by agarose gel electrophoresis and PAGE.

PCR products were purified and recovered from mutant strains, and ligated into T-vectors for sequencing, and mutant materials with knocked-out OsAFB4 gene in rice were obtained.

The primer sequences are shown as follows:

OsLEC1 C-F:
(Sequence XIV)
TGTGGTCAACACCTCGTAGC
OsLEC1 C-R:
(Sequence XV)
GACTTGAGCATCCCACCGAC

PCR reaction system

| Reagents | Volume (μL) |
|---|---|
| Diluted GG product | 2.5 |
| OsLEC1 C-F | 2.5 |
| OsLEC1 C-R | 2.5 |
| 2 × TaqMix | 25 |
| Three-distilled water | 17.5 |
| Total volume | 50 |

PCR procedure

| Temperature (° C.) | Time | Cycle number |
|---|---|---|
| 95 | 5 min | 1 |
| 95 | 30 s | 33 |
| 60 | 30 s | |
| 72 | 30 s | |
| 72 | 10 min | 1 |
| 94 | 3 min | 1 |
| 42 | 30 min | 1 |

The PCR products of the above mutant strains were purified and recovered, and sequenced. The sequencing results are shown in FIGS. 1A and 1B. Sequence analysis revealed that in the target sites of gRNA3 and gRNA4 from lineages 3 # and 4 #, there were deletions of 46 bp and 25 bp, along with a 2 bp deletion and a 1 bp insertion. These sequence alterations were predicted to induce premature termination codons, resulting in truncated proteins of 76 and 35 amino acids in length. At this point, the rice OsLEC1 gene knockout material was obtained. In the figure, gRNA1 and gRNA2 are marked as the knockout sites of two reported lines, Oslec1-1 and -2. The previous knockout sites are located behind the key domain of OsLEC1, and their knockout does not affect the transcription of the key domain part. Therefore, a mutant with the knockout site at the key domain was newly constructed.

As shown in FIGS. 2A and 2B, 30 fresh seeds of WT and Oslec1 mutant were taken, shelled, sterilized with 70% alcohol for 3 min, sterilized with 30% sodium hypochlorite solution for 10 m, rinsed with sterile water for 6 times, spread on callus induction medium, and cultured in a light incubator at 28° C. for about 1 month. When granular callus appeared, the granular callus was subcultured on new callus induction medium for about 20 days to expand propagation, the callus granules with good growth state and similar size were selected and placed on the differentiation medium, and photographs were taken for about 30 days and budding rate was counted. The results showed that the budding rate of callus differentiation of Oslec1 mutant was significantly higher than that of wild type. It was shown that the knockdown of OsLEC1 could promote the differentiation of rice callus.

Formulation of callus induction medium

| Reagents | Dosage | |
|---|---|---|
| N6 medium powder | 3.859 | g |
| 2,4-D (1 mg/mL) | 2 | mL |
| L-proline | 2.8 | g |
| Casein hydrolase | 0.5 | g |
| Sucrose | 30 | g |
| Vegetable gel | 4.5 | g |

| | |
|---|---|
| PH value | 5.8 |

Formulation of callus differentiation medium

| Reagents | Dosage | |
|---|---|---|
| Murashige & Skoog medium (MS) | 4.47 | g |
| powder | | |
| 6-BA (1 mg/mL) | 3 | mL |
| 1-Naphthaleneacetic acid (NAA) | 0.5 | mL |
| (1 mg/mL) | | |
| Sorbitol | 20 | g |
| Sucrose | 30 | g |
| Vegetable gel | 4.5 | g |

| | |
|---|---|
| PH value | 5.5 |

In summary, in the present disclosure, the differentiation of callus directly affects the emergence efficiency of transgenic plants. The knockout of OsLEC1 can promote the differentiation of rice callus, suggesting that OsLEC1 may serve as an important target gene for improving the transformation efficiency of rice and even gramineous crops. OsLEC1 can be used as a starting point to construct various molecular tools to enhance transformation efficiency.

The basic principle, main features and advantages of the present disclosure have been shown and described above. It is to be understood by those skilled in the art that the present disclosure is not limited by the above examples, and the above examples and the specification only describe the principles of the present disclosure. Under the premise of not departing from the spirit and scope of the present disclosure, there will be various changes and improvements to the present disclosure, and all these changes and improvements fall within the scope of the present disclosure for which protection is claimed. The scope of protection claimed by the present disclosure is defined by the attached claims and their equivalents.

>0.sativa v7.0|LOC_Os02g49370|Chr2:30164272..30165441 reverse
Sequence I
CACACTTTAGCTGCTAGCTAGTGCTACCACCCACAGGAGGCAACCCTAGCTTTCCCACGC
AGCCAATGGAGGCCGGCTACCCGGGCACGGCGGCGAACGGCGCTGCCGCCGACGGGAA
CGGTGGCGCGCAGCAGGCGGCGGCCGCGCCGGCTATACGTGAGCAGGACCGGCTGATG
CCGATCGCGAACGTGATCCGCATCATGCGCCGCGTGCTCCCGGCGCACGCCAAGATCTC
GGACGACGCCAAGGAGACGATCCAGGAGTGCGTGTCGGAGTACATCAGCTTCATCACCG
GGGAGGCCAACGAGCGGTGCCAGCGCGAGCAGCGCAAGACCATCACCGCCGAGGACG
TGCTCTGGGCCATGAGCCGCCTCGGCTTCGACGACTACGTCGAGCCCCTCGGCGTCTAC
CTCCACCGCTACCGCGAGTTCGAGGGGGAGTCCCGCGGCGTCGGCGTCGGCGTCGGCG
CCGCGCGCGGCGACCACCACCATGGTCACGTCGGTGGGATGCTCAAGTCCCGCGCGCAG
GGCTCCATGGTGACGCACCACGACATGCAGATGCACGCCGCCATGTACGGTGGCGGCGC
GGTGCCGCCGCCGCCGCATCCTCCTCCGCACCACCACGCGTTCCACCAGCTCATGCCGC
CGCACCACGGCCAGTACGCGCCGCCGTACGACATGTACGGCGGCGAGCACGGGATGGC
GGCGTACTACGGCGGGATGTACGCGCCCGGCAGCGGCGGCGACGGGAGCGGCAGCAGC
GGCAGCGGTGGCGCCGGCACGCCGCAGACCGTCAACTTCGAGCACCAGCATCCGTTCG
GATACAAGTAGTAACAGCAGCAGAATGGCGATCGGTCTGCACCTGCATGCACACGTATC
GCATGCAGATCGAGCTAGTAGTGCAACTGGCTAACTTGAACCAGTTAAAAACTTAAGAC
TTAGTGATCGTGTGTGGTTTAATTAATTTGCTACGTTCAGGTAGTGATCGATGAGATTTTAA
TTTCCTACTGTCAGTTTAATTCACCGTTCATGTACTCGATCCAGCTAGTACTACTCCTAGTT
ACCCTTGTTCTAATCTTAACGCAATTGTGTCGATCGGACGATCGCACGTTTATCTTCAAGT
GGAATTTGTCTTATAACACTAATTAAGCGCCTGGAAGCTTATGTACA
>0.sativa v7.0|LOC_Os02g49370.1 CDS
Sequence II
ATGGAGGCCGGCTACCCGGGCACGGCGGCGAACGGCGCTGCCGCCGACGGGAACGGTG
GCGCGCAGCAGGCGGCGGCCGCGCCGGCTATACGTGAGCAGGACCGGCTGATGCCGATC
GCGAACGTGATCCGCATCATGCGCCGCGTGCTCCCGGCGCACGCCAAGATCTCGGACGA
CGCCAAGGAGACGATCCAGGAGTGCGTGTCGGAGTACATCAGCTTCATCACCGGGGAGG
CCAACGAGCGGTGCCAGCGCGAGCAGCGCAAGACCATCACCGCCGAGGACGTGCTCTG
GGCCATGAGCCGCCTCGGCTTCGACGACTACGTCGAGCCCCTCGGCGTCTACCTCCACC
GCTACCGCGAGTTCGAGGGGGAGTCCCGCGGCGTCGGCGTCGGCGTCGGCGCCGCGCG
CGGCGACCACCACCATGGTCACGTCGGTGGGATGCTCAAGTCCCGCGCGCAGGGCTCCA
TGGTGACGCACCACGACATGCAGATGCACGCCGCCATGTACGGTGGCGGCGCGGTGCCG
CCGCCGCCGCATCCTCCTCCGCACCACCACGCGTTCCACCAGCTCATGCCGCCGCACCA
CGGCCAGTACGCGCCGCCGTACGACATGTACGGCGGCGAGCACGGGATGGCGGCGTACT
ACGGCGGGATGTACGCGCCCGGCAGCGGCGGCGACGGGAGCGGCAGCAGCGGCAGCG
GTGGCGCCGGCACGCCGCAGACCGTCAACTTCGAGCACCAGCATCCGTTCGGATACAAG
TAG
>0.sativa v7.0|LOC_Os02g49370.1
Sequence III
MEAGYPGTAANGAAADGNGGAQQAAAAPAIREQDRLMPIANVIRIMRRVLPAHAKISDDA
KETIQECVSEYISFITGEANERCQREQRKTITAEDVLWAMSRLGFDDYVEPLGVYLHRYREF
EGESRGVGVGVGAARGDHHHGHVGGMLKSRAQGSMVTHHDMQMHAAMYGGGAVPPPP
HPPPHHHAFHQLMPPHHGQYAPPYDMYGGEHGMAAYYGGMYAPGSGGDGSGSSGSGGA
GTPQTVNFEHQHPFGYK

1. An application method in rice callus differentiation based on an Oryza sativa Leafy Cotyledon 1 (OsLEC1) gene, comprising the steps of:
S1, selecting guide ribonucleic acid (gRNA) target sites, the sequence of the target sites being located on the OsLEC1 gene, and the OsLEC1 gene being Sequence I recorded in this invention;
S2, cloning tandem fragments comprising gRNA;
S3, ligating each gRNA fragment;
S4, performing polymerase chain reaction (PCR) amplification on a ligation product;
S5, performing enzyme digestion on the purified product and a target vector;
S6, transforming the ligated vector;
S7, performing Agrobacterium-mediated genetic transformation of rice; and
S8, screening and identifying transgenetic plants.

2. The application method in rice callus differentiation based on an OsLEC1 gene according to claim 1, wherein the selecting gRNA target sites specifically comprises the steps of:
following target site design principles for clustered regularly interspaced short palindromic repeats-CRISPR-associated protein 9 (CRISPR-Cas9) technology; and
designing two gRNA target sites on a gene sequence close to a 5′ end of the OsLEC1 gene.

3. The application method in rice callus differentiation based on an OsLEC1 gene according to claim 2, wherein the cloning tandem fragments comprising gRNA specifically comprises the steps of:
using plasmid pGTR as a template, and amplifying three fragments L1, L2, and L3 comprising gRNA sequences by PCR; and
taking 5-10 μL of products, detecting the products by 1% agarose gel electrophoresis, purifying and recovering a target fragment, and determining the three PCR products L1, L2 and L3.

4. The application method in rice callus differentiation based on an OsLEC1 gene according to claim 3, wherein the ligating each gRNA fragment specifically comprises the steps of:
mixing the 3 fragments in equal amounts according to concentrations of the determined PCR products; and
adding T7 ligase and BsaI enzyme, and performing a reaction in a PCR instrument.

5. The application method in rice callus differentiation based on an OsLEC1 gene according to claim 4, wherein the T7 ligase and BsaI enzyme are reacted in the PCR instrument at 37° C. for 5 min and at 20° C. for 10 min, and cycled for 30-50 times.

6. The application method in rice callus differentiation based on an OsLEC1 gene according to claim 4, wherein the performing PCR amplification on a ligation product specifically comprises the steps of:
taking, after the ligation reaction is completed, 1 μL of the ligation product, and diluting the ligation product with 19 μL of water;
using the diluted product was as a template, and performing PCR amplification with primers at two ends; and
taking, after the PCR is completed, 5 μL of the product for electrophoresis detection, with a product size of 500 bp, and purifying the product.

7. The application method in rice callus differentiation based on an OsLEC1 gene according to claim 6, wherein the performing enzyme digestion on the purified product and a target vector specifically comprises the steps of:
digesting the purified PCR product with FokI, and digesting the target vector pRGEB32 with BsaI; and
mixing, after the digested products are recovered separately, the purified PCR product and the target vector recovered after digestion in equal amounts and ligating the same with T4 ligase at 4° C. overnight.

8. The application method in rice callus differentiation based on an OsLEC1 gene according to claim 7, wherein the transforming the ligated vector specifically comprises the steps of:
transforming the ligated vector into competent cells of Escherichia coli, smearing, and staying overnight at 37° C.;
picking a single colony for bacterial culture, and identifying whether the target fragment is ligated into the vector by PCR; and
identifying the correctness through sequencing after plasmid extraction, and electroporating the correctly sequenced plasmid into Agrobacterium EHA105.

9. The application method in rice callus differentiation based on an OsLEC1 gene according to claim 8, wherein the performing Agrobacterium-mediated genetic transformation of rice specifically comprises the steps of:
using mature embryo callus of wild-type (WT) rice Nipponbare as explants;
performing infection transformation with Agrobacterium EHA105; and
obtaining the transgenetic plants through resistance screening, tissue differentiation, rooting and seedling refining.

10. The application method in rice callus differentiation based on an OsLEC1 gene according to claim 9, wherein the screening and identifying transgenetic plants specifically comprises the steps of:
extracting genomic DNA of transgenetic plants, and designing primers on two sides of two gRNA sequences;
performing PCR amplification on the target fragment, and detect mutants by agarose gel electrophoresis and polyacrylamide gel electrophoresis (PAGE); and
purifying and recovering PCR products from mutant strains, and ligating into T-vectors for sequencing, and obtaining mutant materials with knocked-out Oryza sativa AUXIN SIGNALING F-BOX PROTEIN 4 (OsAFB4) gene in rice.


SEQUENCE LISTING (18 sequences)

SEQ ID NO: 1 - DNA, 1169 bp, Oryza sativa
  source 1..1169: mol_type = genomic DNA; note = Nucleotide sequence of gene OsLEC1
cacactttagctgctagctagtgctaccacccacaggaggcaaccctagctttcccacgcagccaatggaggccggctacccgggcacggcggcgaacggcgctgccgccgacgggaacggtggcgcgcagcaggcggcggccgcgccggctatacgtgagcaggaccggctgatgccgatcgcgaacgtgatccgcatcatgcgccgcgtgctcccggcgcacgccaagatctcggacgacgccaaggagacgatccaggagtgcgtgtcggagtacatcagcttcatcaccggggaggccaacgagcggtgccagcgcgagcagcgcaagaccatcaccgccgaggacgtgctctgggccatgagccgcctcggcttcgacgactacgtcgagcccctcggcgtctacctccaccgctaccgcgagttcgagggggagtcccgcggcgtcggcgtcggcgtcggcgccgcgcgcggcgaccaccaccatggtcacgtcggtgggatgctcaagtcccgcgcgcagggctccatggtgacgcaccacgacatgcagatgcacgccgccatgtacggtggcggcgcggtgccgccgccgccgcatcctcctccgcaccaccacgcgttccaccagctcatgccgccgcaccacggccagtacgcgccgccgtacgacatgtacggcggcgagcacgggatggcggcgtactacggcgggatgtacgcgcccggcagcggcggcgacgggagcggcagcagcggcagcggtggcgccggcacgccgcagaccgtcaacttcgagcaccagcatccgttcggatacaagtagtaacagcagcagaatggcgatcggtctgcacctgcatgcacacgtatcgcatgcagatcgagctagtagtgcaactggctaacttgaaccagttaaaaacttaagacttagtgatcgtgtgtggtttaattaatttgctacgttcaggtagtgatcgatgagattttaatttcctactgtcagtttaattcaccgttcatgtactcgatccagctagtactactcctagttacccttgttctaatcttaacgcaattgtgtcgatcggacgatcgcacgtttatcttcaagtggaatttgtcttataacactaattaagcgcctggaagcttatgtaca

SEQ ID NO: 2 - DNA, 765 bp, Oryza sativa
  source 1..765: mol_type = genomic DNA; note = Coding sequence for gene OsLEC1
atggaggccggctacccgggcacggcggcgaacggcgctgccgccgacgggaacggtggcgcgcagcaggcggcggccgcgccggctatacgtgagcaggaccggctgatgccgatcgcgaacgtgatccgcatcatgcgccgcgtgctcccggcgcacgccaagatctcggacgacgccaaggagacgatccaggagtgcgtgtcggagtacatcagcttcatcaccggggaggccaacgagcggtgccagcgcgagcagcgcaagaccatcaccgccgaggacgtgctctgggccatgagccgcctcggcttcgacgactacgtcgagcccctcggcgtctacctccaccgctaccgcgagttcgagggggagtcccgcggcgtcggcgtcggcgtcggcgccgcgcgcggcgaccaccaccatggtcacgtcggtgggatgctcaagtcccgcgcgcagggctccatggtgacgcaccacgacatgcagatgcacgccgccatgtacggtggcggcgcggtgccgccgccgccgcatcctcctccgcaccaccacgcgttccaccagctcatgccgccgcaccacggccagtacgcgccgccgtacgacatgtacggcggcgagcacgggatggcggcgtactacggcgggatgtacgcgcccggcagcggcggcgacgggagcggcagcagcggcagcggtggcgccggcacgccgcagaccgtcaacttcgagcaccagcatccgttcggatacaagtag

SEQ ID NO: 3 - protein, 254 aa, Oryza sativa
  source 1..254: note = amino acid sequence coded by nucleotide sequence 2, coding sequence of gene OsLEC1
MEAGYPGTAANGAAADGNGGAQQAAAAPAIREQDRLMPIANVIRIMRRVLPAHAKISDDAKETIQECVSEYISFITGEANERCQREQRKTITAEDVLWAMSRLGFDDYVEPLGVYLHRYREFEGESRGVGVGVGAARGDHHHGHVGGMLKSRAQGSMVTHHDMQMHAAMYGGGAVPPPPHPPPHHHAFHQLMPPHHGQYAPPYDMYGGEHGMAAYYGGMYAPGSGGDGSGSSGSGGAGTPQTVNFEHQHPFGYK

SEQ ID NO: 4 - DNA, 46 bp, synthetic construct
  source 1..46: mol_type = other DNA; note = Nucleotide sequence of primer LI-F
cgggtctcaggcaggatgggcagtctgggcaacaaagcaccagtgg

SEQ ID NO: 5 - DNA, 34 bp, synthetic construct
  source 1..34: mol_type = other DNA; note = Nucleotide sequence of primer LI-R
cgggtctcagcgtgcgccgggtgcaccagccggg

SEQ ID NO: 6 - DNA, 36 bp, synthetic construct
  source 1..36: mol_type = other DNA; note = Nucleotide sequence of primer L2-F
taggtctccacgccaagatctgttttagagctagaa

SEQ ID NO: 7 - DNA, 34 bp, synthetic construct
  source 1..34: mol_type = other DNA; note = Nucleotide sequence of primer L2-R
cgggtctcatagccggcctcctgcaccagccggg

SEQ ID NO: 8 - DNA, 36 bp, synthetic construct
  source 1..36: mol_type = other DNA; note = Nucleotide sequence of primer L3-F
taggtctccgctacccgggcagttttagagctagaa

SEQ ID NO: 9 - DNA, 52 bp, synthetic construct
  source 1..52: mol_type = other DNA; note = Nucleotide sequence of primer L3-R
taggtctccaaacggatgagcgacagcaaacaaaaaaaaaagcaccgactcg

SEQ ID NO: 10 - DNA, 31 bp, synthetic construct
  source 1..31: mol_type = other DNA; note = Nucleotide sequence of primer S1-F
cgggtctcaggcaggatgggcagtctgggca

SEQ ID NO: 11 - DNA, 31 bp, synthetic construct
  source 1..31: mol_type = other DNA; note = Nucleotide sequence of primer S1-R
taggtctccaaacggatgagcgacagcaaac

SEQ ID NO: 12 - DNA, 22 bp, synthetic construct
  source 1..22: mol_type = other DNA; note = Nucleotide sequence of primer U3-F
agtaccacctcggctatccaca

SEQ ID NO: 13 - DNA, 20 bp, synthetic construct
  source 1..20: mol_type = other DNA; note = Nucleotide sequence of primer UGW-gRNA-R
cgcgctaaaaacggactagc

SEQ ID NO: 14 - DNA, 20 bp, synthetic construct
  source 1..20: mol_type = other DNA; note = Nucleotide sequence of primer OsLEC1 C-F
tgtggtcaacacctcgtagc

SEQ ID NO: 15 - DNA, 20 bp, synthetic construct
  source 1..20: mol_type = other DNA; note = Nucleotide sequence of primer OsLEC1 C-R
gacttgagcatcccaccgac

SEQ ID NO: 16 - DNA, 60 bp, Oryza sativa
  source 1..60: mol_type = genomic DNA; note = Nucleotide sequence of PCR product from Oryza sativa, WT
caatggaggccggctacccggtgcacgccgcgtgctcccggcgcacgccaagatctcgga

SEQ ID NO: 17 - DNA, 60 bp, Oryza sativa; identical to SEQ ID NO: 16
  source 1..60: mol_type = genomic DNA; note = Nucleotide sequence of PCR product from Oryza sativa, strain 3#

SEQ ID NO: 18 - DNA, 61 bp, Oryza sativa
  source 1..61: mol_type = genomic DNA; note = Nucleotide sequence of PCR product from Oryza sativa, strain 4#
caatggaggccggctacccggtgcacgccgcgtgctcccggcgcacgccaagaatctcgga